(19) United States
(12) Patent Application Publication
(10) Pub. No.: US 2018/0110946 A1
(43) Pub. Date: Apr. 26, 2018

(54) AIR IMPELLER DEVICE FOR PROVIDING ASSISTED VENTILATION DURING SPONTANEOUS BREATHING

(71) Applicant: TÉCNICAS BIOMÉDICAS PARA LA SALUD, S.L., Sant Adrià Del Besòs (ES)

(72) Inventor: Jaume PALOU FUSTÈ, Sant Adrià Del Besòs (ES)

(21) Appl. No.: 15/564,975

(22) PCT Filed: Apr. 7, 2016
(86) PCT No.: PCT/EP2016/057575

§ 371 Date: Oct. 6, 2017

(30) Foreign Application Priority Data
Apr. 7, 2015 (ES) P201530458

(51) Int. Cl. A61M 16/00; A61M 16/06; A61M 16/16; A61M 16/08; A62B 18/00

(52) U.S. Cl. CPC A61M 16/0069; A61M 16/06; A61M 2016/1025; A61M 16/0816; A62B 18/006; A61M 16/161

(57) ABSTRACT

Air impeller devices for providing assisted ventilation during spontaneous breathing are described. The devices include a motor; a fan driven by the motor, and a casing defining a housing for the fan. The housing is connectable through a single inlet and outlet port to a respiratory mask, wherein a pressure inside the housing is adjustable according to a rotational speed of the fan such that, in use, an inspiration air flow and an exhalation air flow circulate substantially through the inlet and outlet port. A kit including such an air impeller device and a respiratory mask is also disclosed.

The present disclosure relates to air impeller devices for providing assisted ventilation during spontaneous breathing, for use, for example, during sleep.

### BACKGROUND

Continuous positive air pressure devices (CPAP) are known to assist breathing of a patient whose lung capacity is diminished. These devices are thus applicable, for example, to patients with obstructive sleep apnea (OSA) and other breathing disorders, such as resistance to air flow in the upper airways that causes snoring. CPAP devices are mainly used for supplying air, or other breathable gas under pressure to the airways of a patient.

Conventional CPAP devices generally comprise a compressed air generator of the blower type which is arranged, for example, in the night table near the bed and is connected to the electrical grid or to a large external battery. Furthermore, the compressed air generator is usually connected to the patient through a tube or a long and flexible duct (corrugated or not), commonly known as trachea, with a variable length (depending on circumstances, e.g. from about 0.5 to 2 m. length) carrying the pressurized air from the air flow generator to an interface with the patient, for example a respiratory mask worn by the patient. Such a supplied air prevents episodes of collapse of the upper airways that block breathing in persons with OSA and other respiratory disorders.

Throughout the present description and claims, a compressed air generator of the blower type for CPAP should be understood as a centrifugal pump with a propeller comprising blades with output angle lower than 90°. This way, the pressure/flow relationship of the device is inherently stable. This means that, for a given rotational speed, an increase in the flow rate means a monotonic decrease in pressure and vice versa. This behavior is desirable when it is intended to ensure that the air flows in a single direction in a stable manner. This behavior is normally used by all manufacturers of CPAP with air generator of the blower type. Such pumps work in the first quadrant of its operating characteristic curve therefore supplied flow and pressure are both positive. Additionally, in these systems clearances connecting the inlet and outlet need to be minimized in order to prevent flow losses due to recirculation. Also, in these systems, the mobile elements (e.g. blades) typically rotate at high speed thus involving a large amount of stored kinetic rotation energy in spite of its mass being usually low. Therefore, changes in the regime of the motor are usually quite inertial and slow.

Respiratory masks usually used can comprise a nasal mask designed to fit over the nose of a patient, or a full face mask designed to fit over the nose and mouth of the patient. It is desirable that this setting is done safely to deliver compressed air without substantial leakage. And known CPAPs usually have necessary vents either in the mask or in its connecting elbow. This involves great air flow losses, and therefore power, both during inspiration and exhalation. This results in supplied air flows becoming even double the actual user demand. Such vents are necessary due to the inability to exhale (breathe) through the tube itself thereby resulting in rebreathing stale air. Additionally, air flow always flows from the drive member to the mask.

These respiratory masks generally comprise a relatively rigid portion defining a cavity with backwards opening and covering the nose and/or the nose and mouth of the patient, and a soft portion, for example a pad, which separates the rigid portion from the face of the patient so as to make contact comfortable. Furthermore, they use to be held in place by, for example, straps attached to the rigid portion by connectors.

Known conventional CPAP devices are thus often too bulky and heavy, making them cumbersome to transport for people requiring its use. They also need to generate air at a high enough pressure to be able to run along the entire tube connecting the air flow generator with the mask, overcoming internal resistances of the tube. This usually brings with it considerable noise levels, especially annoying when these systems are used during sleep.

Currently, in order to reduce internal resistances of the tube connecting the air flow generator to the mask, CPAP devices comprising a mask and an air flow generator mounted on the mask or adapted to be coupled to the body of the user have been developed, i.e. they are connected to the mask by a tube with a length below 2 meters long or even with a length of up to half a meter long. U.S. Pat. No. 8,844,524 describes such devices in which it is possible to reduce, at least partially, air resistance inside the tube, thereby also reducing, at least in part, some of the above mentioned drawbacks. However, these systems need to be connected to the electrical grid or to a large external battery.

Thus, there remains a need to develop CPAP devices which are more versatile, less noisy and give the user a greater degree of mobility to improve their life quality.

### SUMMARY

In an aspect, an air impeller device for providing assisted ventilation by nasal mask during spontaneous breathing is provided. The device comprises a motor, a fan driven by the motor, and a casing defining a housing for the fan. The housing is connectable via a single inlet and outlet port to the respiratory nasal mask, wherein a pressure and air flow circulation direction inside and through the housing is adjustable as a function of a rotational speed of the fan such that, in use, an inspiration air flow and an exhalation air flow circulate substantially through the single inlet and outlet port and through the fan.

According to this aspect, in use, i.e. when the housing is connected (in fluid communication) to a respiratory nasal mask, and the respiratory nasal mask is, in turn, adjusted or coupled to a patient, the inspiration air flow driven by the ventilator and the exhalation air flow caused by the patient flow through the same inlet and outlet port. Such an exhalation air flow also circulates through the fan. This is possible because the pressure and air flow circulation direction inside the housing is adjusted. Such an adjustment is done by increasing or decreasing the rotational speed of the fan depending if it is inspiration air flow or exhalation air flow respectively, enabling the reversal of the air flow direction without stopping the motor to change its direction of rotation. During exhalation the fan is thus working in the second quadrant of the characteristic flow/pressure, i.e. positive pressure and negative flow. This involves that, at the time of exhalation (simultaneously) the power consumed by the motor is significantly reduced. This reduction in the power consumed by the motor during patient exhalation may be detected by the electronics controlling the motor which, in turn, reduces the rotational speed.

In addition, by allowing the passage of exhalation air flow through the fan and through the same inlet and outlet port as the inspiration air flow eliminates the presence of an exhausting exit, normally provided in masks or in some portion of the inlet and outlet port for exiting of the exhalation air. This results in a reduced air flow driven by the ventilator since this flow driven by the ventilator can only follow its way to be inspired by the patient. Put in other words, the air blown by the ventilator does not divide (branch) in two (or more) paths, one towards the nose and/or nose and mouth of the patient and the other towards the exhaust(s) exit(s). Such a flow rate of air blown by the ventilator reduction involves lower energy consumption by the ventilator, which allows the use of smaller power sources such as for example a standard battery or converter of 2 A and 5V. This considerably enhances the portability of the devices providing greater autonomy to the user. It also entails a significant reduction in noise levels, thus improving life quality of the users.

Throughout the present description and claims it should be understood that a nasal mask is a mask covering either only the nose or mouth and nose of a patient.

In some examples, the reduced fan speed can be achieved by disconnecting a servomechanism that maintains stable the supplied pressure and recovering the rotational kinetic energy of the moving parts (e.g. the blades) by an electronic brake.

In some examples, the fan may comprise a plurality of blades whose output angle may be greater than 90°. This output arrangement of the blades enhances the effect of reversing flow, i.e. improves passage of inspiratory air flow and exhalation air flow through the fan itself without reversing its direction of rotation. It is thus only necessary to regulate the speed of rotation of the fan. As described above, in these examples, the fan works, during exhalation, in the second quadrant of the characteristic flow/pressure, i.e. positive pressure and negative flow.

The arrangement of the blades with an output angle higher than 90° enhances the effect of flow reversal since it produces an intrinsic instability in the response flow/pressure. This inherent instability on the one hand may be compensated during inhalation with a measurement of the pressure inside the housing. Such a pressure measurement may act as a control variable in the servomechanism that regulates the motor speed, thus working at constant pressure during inhalation. On the other hand, during exhalation this modus operandi (at a constant pressure) may be disconnected thereby allowing operation to be governed by the driving force of the patient's lungs which can balance the air flow due to the unstable characteristic of the flow through the blower type fan.

In some examples, the inlet and outlet port may be configured to be connected to a mask without additional ventilation exit. This ensures no branches of the inspiration air flow (air driven by the ventilator) and that exhalation air flow can only circulate through the inlet and outlet port of the device like the inspiration air flow.

In some examples, the inlet and outlet port may comprise a coupling element, for example a frustoconical coupling. This type of coupling allows a tongue and groove coupling with practically any commercially available mask having a feeding port with a cylindrical or truncated cone inlet/outlet that is complementary to the single inlet and outlet port.

According to some examples, the inlet and outlet port may comprise circular sections whose diameter may be between 10 mm and 40 mm. In some of these cases, a tolerance of about 10%, depending on the elastic properties of the material with which the ports of the mask and/or the impeller device were manufactured may be necessary.

According to further examples, the inlet and outlet port may be a piping/plumbing fitting (racor) made of polymeric material. The material may be any solid material having elastic properties such as, for example, rubber, caoutchouc, silicone or the like.

According to some examples, the fan may drive the flow of inspiration air at a pressure such that in use, inside the mask, the inspiration air flow may have a pressure of between 0 and 30 cm H₂O.

In some examples, the ventilator may be a radial fan. The inventors have found that these type of ventilators involve the largest reductions in noise, thus enhancing life quality of the users. In other examples, axial fans or centrifugal fans may also be used.

In some examples, the ventilator may be mounted within the housing (or part of the housing) by a clip-coupling system which allows easy assembly and disassembly. This allows a relatively quick dismantling and facilitates cleaning of the fan. In more examples, the device may include one or more UV light emitting diodes (LEDs) arranged to illuminate an inside of the inlet and outlet port as well as the fan. Illumination with ultraviolet light provides sterilization in these areas. Cleaning and sterilization of the fan and of the inlet and outlet port are especially interesting due to the dual circulation (inspiration air flow and exhalation air flow) to which it is subjected. It is important to note that exhalation air flow usually comprises a greater number of contaminant particles and humidity.

In some examples, the device may further comprise an electronic control and power supply board attachable to the casing. The fact that it is attachable to, and detachable from, the casing, enables complete dismantling of the mechanical part from the electronics controlling the device, which facilitates cleaning of the mechanical part (the fan and the inlet and outlet port), which mechanical part can be washed even in a domestic dishwasher.

In some examples, the electronic control and power supply board may comprise a communication interface such as a USB port (for example in versions 2.0 or 3.0) or Bluetooth and/or a power supply port attachable to a battery or a standard power converter, e.g. of 2 A and 5V. This type of energy supply is possible due to the reduction in the air flow driven by the fan substantially as hereinbefore described. Because the inspiration air flow circulates practically through a single inlet and outlet port (i.e., it is not divided, for example to an exhaust exit), losses of the air flow driven by the fan are substantially reduced thus reducing substantially (at least partially) the power required to operate it. In addition, through the USB port or Bluetooth, the device may be connected to a blood oxygen meter or it may control a working regime of the blower type fan, as well as a spray (aerosol) supply. This is particularly interesting in cases of pulmonary diseases. In some examples, the data may be stored and monitored.

In more examples, the electronic control and power supply board may comprise a microprocessor that allows, in its memory, for example, to record and store values measured by sensors. This information is useful for the specialist that analyses the clinical condition of the patient. In addition, this information is easily extractable from the memory to a computer through the same USB port or Bluetooth (or any other type of communication interface) provided on the board. In these examples, the device may comprise one or more buttons configured to activate different operational options such as fan operation or “flight mode”, with antenna disconnected, commonly used in any Bluetooth device. In some examples, the device may comprise one or more LEDs, the LEDs including one or more colors and being governed by the microprocessor. The LEDs may indicate the states of the device and may apply light stimuli to the patient.

According to some examples, the device may further comprise one or more sensors selected from the group consisting of CO₂ sensors, O₂ sensors, temperature sensors, acceleration sensors, pressure sensors, humidity sensors and flow sensors, the sensors being attachable directly to the inlet and outlet port. This way, the precision with which the ventilator rotational speed is controlled is improved. Providing one or more of these sensors enhances detection of exhalation air flow (exhaled) either because, for example, an increment in the CO₂ concentration is detected or because an increase in temperature is detected. Alternatively, with a combination of the parameters measured by two or more of these sensors it is possible to further increase the precision with which the rotational speed of the ventilator is regulated to adapt the system to the respiratory rhythm of each patient, i.e. to the inspiration air flow and the exhalation air flow. In some examples, the acceleration sensor may be used to determine the orientation of the head of the patient with respect to the vertical. This way, the air pressure can be modulated in accordance with the degree of obstruction of the soft palate. In addition, the position of the head can be recorded and compared with the incidence of apneas, and finally a change in the position of the head can be forced by acoustic or light stimuli.

In some examples, the sensors may comprise an adaptive control “bi-level”. Thus, the duration of each phase (inspiration/exhalation) with its corresponding pressure level may be adjusted independently.

In some examples, the sensors may be connected to the electronic control and power supply board.

In another aspect, a kit comprising a respiratory nasal mask and an air impeller as described above, coupled to the nasal mask through the single inlet and outlet port is provided. According to some examples, the kit may comprise a non-vented nasal mask. In further examples, when the masks comprise one or more vent openings it is expected that they can be blockable or sealable.

According to some examples, the pressure inside housing of the impeller device may be adjustable according to a rotational speed of the fan such that, in use, an inspiration air flow and an exhalation air flow can circulate substantially only through the single inlet and outlet port of the impeller device.

In some examples, the mask may comprise one or more conductive bands as an electrode arranged in a region of the mask, configured to fit the patient. I.e., at their contact points with, e.g. the patient forehead or face, or a fastening band around the head. This enables measuring the heart rhythm of the patient and its brain activity.

In some examples, the inlet and outlet port may be provided with an access port for, e.g. a nebulizer for delivering aromatic compounds and/or medicines. This enables the application of respiratory therapies that combine these compounds with changes in the working regime of the fan during sleep. In some of these examples, therapies can be adjusted depending on the various parameters that the device can measure: brain activity, existence of apneas or air flow interruptions, inhalation and exhalation flows, depth and rhythm of breathing, heart rhythm, ECG, temperature, inhalation and exhalation pressure, emission of different respiratory sounds. In a particular example, it may be used to apply Ventolin to open airways provided the heart rate does not increase over a prescribed value.

In some examples, the device may comprise a microphone and/or loudspeaker. This enables cancelling, at least in part, the noise generated by the moving and vibrating parts or by user breathing or potentially snoring due to generating a wave in antiphase with any of these noises. Further, in these examples, the device may evaluate with a microphone signal respiratory noises as well as it may generate sound stimuli with the speaker.

In addition, the measurement of the sounds generated by moving and vibrant parts of the device enables to assess the degree of wear of the device and warn of possible preventive or corrective maintenance tasks.

The measurement of the patient's vital signs, especially the heart rate (and cardiac arrest) may be used to trigger alarms either through the loudspeaker that in examples may be incorporated into the electronic of the device or also through its interface in other remote communications devices.

### BRIEF DESCRIPTION OF THE DRAWINGS

The following particular embodiments of the present disclosure by way of non-limiting example described with reference to the accompanying drawings, in which:

FIGS. 1a and 1b show two different perspective views of an air impeller device according to an example;

FIG. 2 shows a side view of FIG. 1b;

FIG. 3 shows a partial cross-section of the impeller device of FIG. 1a;

FIG. 4 shows a scheme of the fan with its blades according to an example; and

FIG. 5 shows an example of a device with a nebulizer.

### DETAILED DESCRIPTION OF EXAMPLES

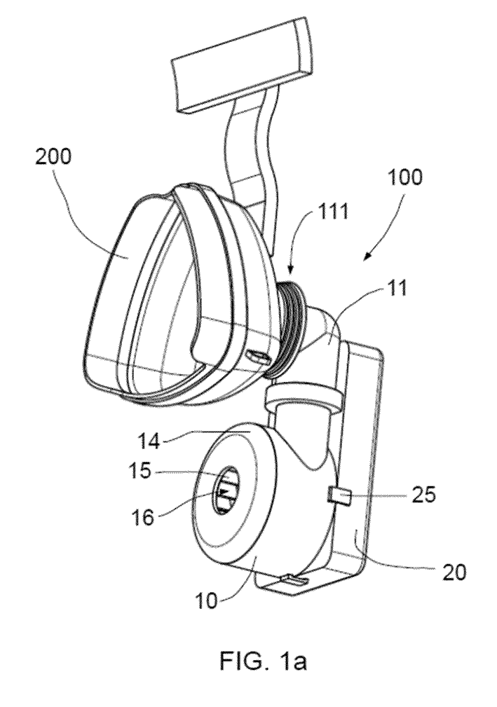

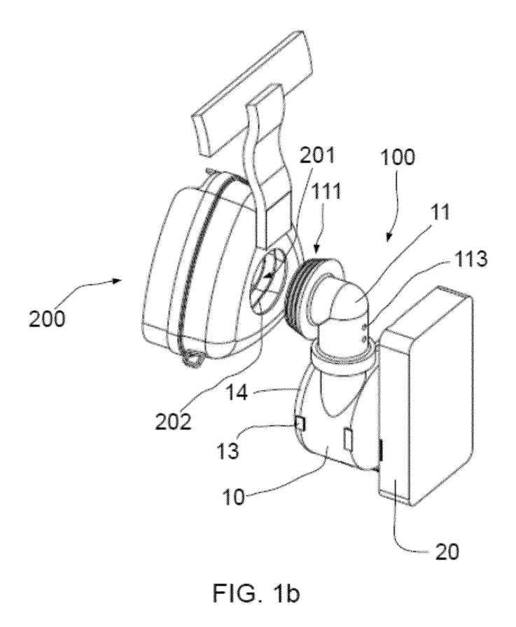

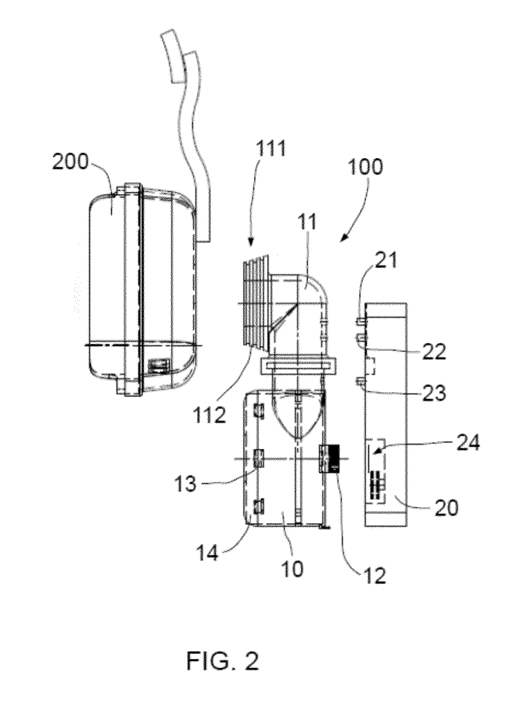

FIGS. 1a and 1b show two perspectives of an air impeller device 100 according to an example. FIG. 2 shows a side view of the same example. In FIGS. 1b and 2 the air impeller device 100 is shown disengaged from a respiratory nasal mask 200 and also disengaged from an electronic control and power supply board 20. In FIG. 1a, instead, the electronic control and power supply board 20 is shown coupled to the impeller device 100.

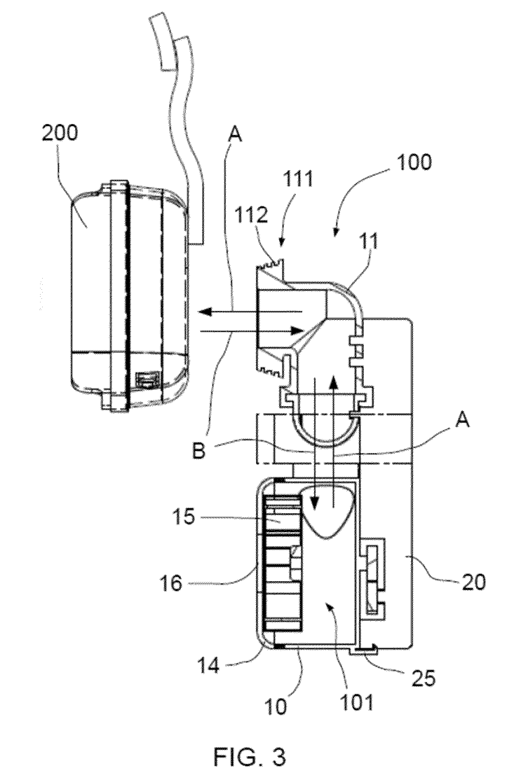

The impeller device 100 may comprise a fan 15 provided inside a housing (reference 101 in FIG. 3) that may be defined by a casing 10. The fan may be driven by a motor (not visible) that may, in turn, be located in the electronic board 20. The device 100 may comprise a single inlet and outlet port 11 attachable to the respiratory mask 200. In some examples, the inlet and outlet port 11 may have a piping/plumbing fitting shape and its free end 111 may comprise a frusto-conical end which, in turn, may comprise a plurality of annular protrusions 112. In alternative examples, other types of protrusions may be provided, such as screw shaped or axially shaped, or even also including discrete protruding points or bayonet connection. Furthermore, the free end 111 of the inlet and outlet port 11 (in the example of the figures, piping/plumbing fitting) may be complementary and/or may permit coupling with an engagement member 201 provided on the respiratory nasal mask 200 adjustable to a patient.

In more examples, the free end of the inlet and outlet port may have internal projections, as long as the coupling element of the mask is external or vice versa. As shown in the example of the figures, the free end 111 of the piping/plumbing fitting may have a frusto-conical shape. Such a shape allows the coupling to be easily combined with, for example, a cylindrical coupling (provided on the mask). This enhances coupling capacity of the impeller device, with almost any existing respiratory nasal mask on the market. This versatility allows the user to use a mask with which the user was already familiar and “comfortable” and couple it to an air impeller device substantially as hereinbefore described. The frusto-conical shape gives good results also in terms of sealing and axial stability between the two components It should be noted that in case the mask worn by the patient comprises some output port for exhalation air flow, this port should be capable of being sealed off or simply covered, so that leakages of air flow driven by the fan (inspiration air) are reduced. This way practically all the air driven by the fan may be inspired by the user, thus preventing part of the air driven by the fan to bifurcate into, e.g. such a vent opening. The passage of both flows (inspiration and exhalation air flow) through the same port is possible because the fan rotational speed is controlled.

In the example of the figures, the free end 111 of the piping/plumbing fitting may comprise a frustoconical shape with annular projections 112, whereas the engagement member 201 of the mask 200 may comprise a cylindrical recess with at least one annular projection 202 for a tongue and groove coupling with at least one of the annular projections 112 of the piping/plumbing fitting. In more examples, other similar coupling elements which result in a tongue-and-groove type coupling between the respiratory mask and the inlet and outlet port of the air impeller device may also be foreseen.

In the example of FIG. 1b, it is also shown that the piping/plumbing fitting may comprise two holes 113 configured to each receive a sensor. In FIG. 2 it is shown that in this example, the electronic control and power supply board 20 may comprise three sensors 21, 22, 23. Sensors 21 and 22 may be coupled in the piping/plumbing fitting holes 113. Sensor 23 may be coupled in another hole (not shown) provided in the piping/plumbing fitting or in the casing 10. In further examples, other number of sensors may be provided configured to be coupled to the inlet and outlet port 11 or the casing 10. In yet further examples, a single sensor or no sensors at all may be foreseen.

As described above, the sensors may be selected from the group consisting of CO₂ sensors, O₂ sensors, temperature sensor, pressure sensors, humidity sensors, noise or microphones and flow sensors. Such sensors improve accuracy and speed with which it is detected whether the air flow flowing through the inlet and outlet port is inspiration or exhalation air flow. Typically, one or more of these parameters (CO₂, O₂, temperature, pressure, humidity, noise) measured by the sensors selected from this group vary considerably depending on whether it is air driven by the fan (inspiration air flow) or exhalation air flow (air exhaled by the patient). In addition, the provision of one or more of these sensors allows defining a breathing rate for each patient. This information may, in turn, be inserted into a microprocessor to set the fan rotational speed according to a preset breathing rate for a patient, for example, to set overnight use. In some cases, the microprocessor may comprise a memory which can store all the information provided by the sensors. This information may be useful to the practitioner that controls the health of the patient. Downloading information may, in turn, be carried out real-time or delayed through the USB port(s) or Bluetooth provided on the control board. This information may be used to locally or remotely monitor the health state of the patient and the degree of adherence to treatment. In other examples, the microprocessor may be configured to receive information from one or more sensors and make decisions on the operation of the fan from this information, i.e., increase or decrease rotational speed of the fan.

Furthermore, in some examples, the sensors may comprise an adaptive “bi-level” control. Such a control allows adjusting independently the duration of each phase (inspiration/exhalation) with its corresponding pressure level/fan rotational speed.

In the example of FIG. 2, it is shown that the casing 10 may comprise a gear 12 (or other known coupling element) for mounting the electronic control and supply board 20 in the casing 10. The electronic board 20 may, in turn, be provided with another coupling of the type of a gear 24 or the like, complementary to the gear 12 or other coupling element provided on the casing. In addition, the electronic board 20 and the casing 10 may be adjusted by means of clip-type couplings 25. Such couplings allow the assembly and disassembly of the electronic board in the casing, for example, to perform cleaning tasks. They therefore allow the fan housed inside the casing to be cleaned, for example, in a dishwasher.

In addition, the casing 10 may be provided with clips 13 or other coupling system for mounting, for example, a cover 14. The cover may be, in turn, provided with a grating or other type of air inlet 16 to input the fan 15 that is housed within the casing 10. The air inlet 16, in turn, may comprise an air filter (not shown), for example, porous consumable material and/or moistenable, to prevent access of particles or impurities present in the air into the fan, as well as providing the patient with a control on the degree of humidity present in the air.

FIG. 3 shows a partial cross-section of FIG. 1a in which the air impeller device 100 is shown disengaged from the respiratory mask 200, but coupled to the electronic control and power supply board 20. In this figure, arrows depict the path of an inspiration air flow (arrow A) and of an exhalation air flow (arrow B). The figure shows the both air flows run practically only through the single inlet and outlet port (11).

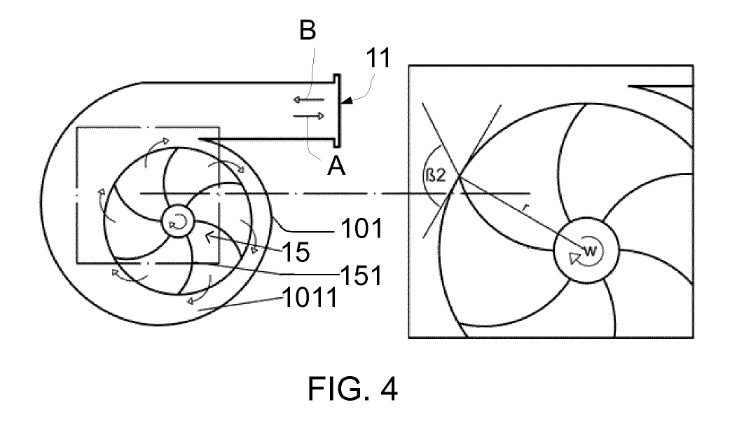

FIG. 4 shows a scheme of an example of the fan 15 disposed within the housing 101. In this figure the inspiration air flow (arrow A) and exhalation air flow (arrow B) are also shown, both circulate through the fan 15 and the single inlet and outlet port 11. FIG. 4 further shows that the fan 15 may comprise a plurality of blades 151. Particularly in this figure six blades are shown, but any other number of blades may also be foreseen. In this example, the blades 151 may be curved forward with respect to its direction of rotation.

According to the enlarged detail of FIG. 4, in these examples, the incident angle (ß2) of the blades may be equal or higher than 90° and less than 180°.

In addition, as further shown in this example, the housing 101 may comprise a spiral shape 1011. Such a shape enhances air circulation as a spiral-shaped perimeter facilitates collection of exhaust air from the fan.

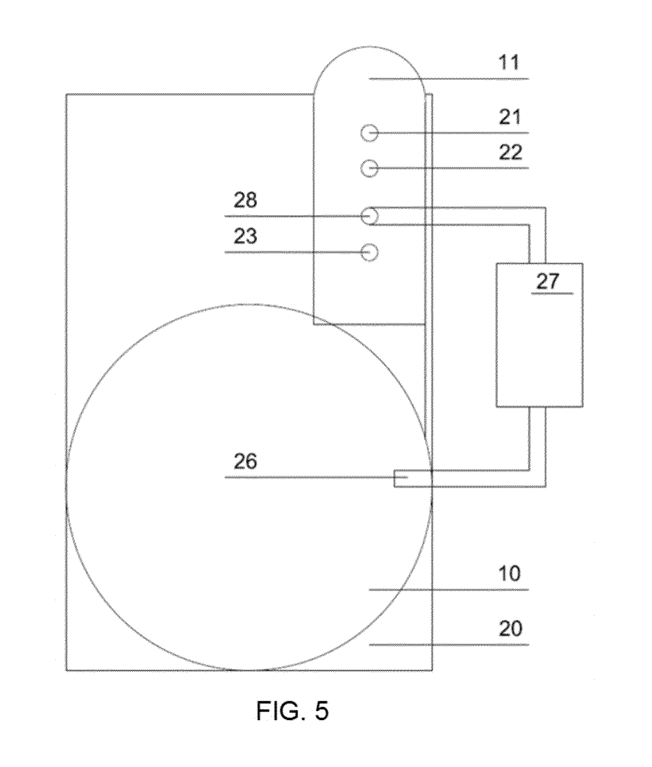

FIG. 5 shows an example of a device with nebulizer. This example shows that the housing 10, the electronic control and power supply board 20 and the single inlet and outlet port 11 may be secondarily connected through an additional element, for example a nebulizer 26. Examples of nebulizers may comprise, e.g., a “venturi” based nebulizer. The nebulizer 26 may comprise an air inlet into the nebulizer 26, a chamber 27 where the air may be mixed with a liquid (not shown), and may be connected through an aperture 28 further provided at the single inlet and outlet port 11, enabling a controlled application of sprays (aerosol) for treatments during sleep. Further, in this example, it is shown that the electronic control and power supply board 20 may comprise three sensors 21, 22, 23. Alternatively, other number of sensors may be foreseen.

Although only a number of examples have been disclosed herein, other alternatives, modifications, uses and/or equivalents thereof are possible. Furthermore, all possible combinations of the described examples are also covered. Thus, the scope of the present disclosure should not be limited by particular examples, but should be determined only by a fair reading of the claims that follow.

1. An air impeller device for providing assisted ventilation by a nasal mask during spontaneous breathing comprising:
a motor;
a fan driven by the motor; and
a casing defining a housing for the fan, the housing being connectable through a single inlet and outlet port to a respiratory nasal mask,
wherein a pressure and an airflow circulation direction inside and through the housing-are adjustable according to a rotational speed of the fan such that, in use, an inspiration air flow and an exhalation air flow circulate substantially through the single inlet and outlet port and through the fan.

2. The device of claim 1, wherein the fan comprises a plurality of blades, the blades comprising an outlet angle higher than 90°.

3. The device of claim 1, wherein the single inlet and outlet port is configured to be connected to a mask that does not have vent openings.

4. The device of claim 1, wherein the single inlet and outlet port comprises a frusto-conical coupling.

5. The device of claim 1, wherein the single inlet and outlet port comprises circular sections whose diameter ranges between 10 mm and 40 mm.

6. The device of claim 1, wherein the single inlet and outlet port is a piping/plumbing fitting made of polymeric material.

7. The device of claim 1, wherein the fan drives the inspiration air flow at a pressure such that, in use, inside the mask, the inspiration air flow has a pressure ranging between 0 and 30 cm H₂O.

8. The device of claim 1, wherein the fan is a radial fan.

9. The device of claim 1, wherein the fan is mounted within the housing by a coupling system made of clips that allow dismantling of the fan from the housing.

10. The device of claim 1, further comprising an electronic control and power supply board configured to engage with the casing.

11. The device of claim 10, wherein the electronic control and power supply board comprises a communication interface.

12. The device of claim 10, wherein the electronic control and power supply board comprises a microprocessor.

13. The device of claim 1, further comprising one or more sensors selected from the group consisting CO₂ sensors, O₂ sensors, temperature sensors, pressure sensors, humidity sensors and flow sensors, the sensors being attachable to the single inlet and outlet port.

14. The device of claim 13, wherein the one or more sensors comprise an adaptive “bi-level” control.

15. The device of claim 13, wherein the one or more sensors are connected to the electronic control and power supply board.

16. The device of claim 1, wherein the single inlet and outlet port is provided with an inlet port for coupling a nebulizer.

17. The device of claim 10, wherein the electronic control and power supply board comprises a microphone and/or a loudspeaker.

18. The device of claim 1, wherein the housing comprises a spiral shape.

19. A kit comprising a respiratory nasal mask and an air impeller device according to claim 1, wherein the air impeller device is coupled to the respiratory nasal mask through the single inlet and outlet port.

20. (canceled)

21. The kit of claim 19, wherein the pressure inside the housing is adjustable according to a rotational speed of the fan such that, in use, an inspiration air flow and an exhalation air flow circulate substantially only through the inlet and outlet port.
